(19) United States
(12) Patent Application Publication
(10) Pub. No.: US 2026/0004364 A1
(43) Pub. Date: Jan. 1, 2026

(54) SYSTEMS AND METHODS FOR AUTOMATIC PAYMENT POSTING

(71) Applicant: The PNC Financial Services Group, Inc., Pittsburgh, PA (US)

(72) Inventors: Deirdre Mary GANNON, Pittsburgh, PA (US); Marina FERRARIS, Pittsburgh, PA (US); Kevin WISNIEWSKI, Pittsburgh, PA (US); Manuel ESTRADA, Pittsburgh, PA (US); Michael C. OLSON, Pittsburgh, PA (US); Elisabeth M. LONNETT, Pittsburgh, PA (US)

(73) Assignee: The PNC Financial Services Group, Inc., Pittsburgh, PA (US)

(21) Appl. No.: 19/234,752

(22) Filed: Jun. 11, 2025

Related U.S. Application Data
(60) Provisional application No. 63/665,442, filed on Jun. 28, 2024.

(51) Int. Cl. G06Q 40/12; G06Q 30/04; G16H 10/60

(52) U.S. Cl. CPC G06Q 40/128; G06Q 30/04; G16H 10/60

(57) ABSTRACT

Computer implemented systems and methods for providing automated payment processing and posting are provided. The systems and methods may include receiving a payment from a third party, receiving a payment statement from the third party, automatically reassociating the payment and the payment statement based on patient data in the payment and the payment statement, and automatically posting, by a robotic process automation platform, the reassociated payment and payment statement to a patient ledger of a practice management software.

### CROSS REFERENCE TO RELATED APPLICATIONS

This application is based on and claims the benefit of U.S. Provisional Application No. 63/665,442, filed Jun. 28, 2024, the contents of which are incorporated herein by reference in their entirety.

### TECHNICAL FIELD

The present disclosure relates to the field of payment posting technology. More specifically, the present disclosure relates to providing an automated payment processing and posting platform.

### BACKGROUND

Revenue cycle management (“RCM”) is a financial process used in the healthcare industry to track patient care from appointment scheduling to final payment to facilitate collection and management of revenue from patient appointments. The RCM process may include scheduling and registration of patients, insurance verification, online payments, insurance claim submissions, payment remittance, explanation of benefits (“EOB”) and electronic remittance advice (“ERA”) to payment reassociation, and payment posting, among other steps. Dental and physician practices typically use a manual process for payment remittance, EOB/ERA and payer payment reassociation, and payer payment posting. For example, practices may receive payer payments in different formats, such as through paper checks, electronic fund transfers (“EFTs”) to a bank account, or through credit card payments. Practices must manually track these payments, deposit paper checks, and process credit card payments. Further, practices may receive paper or electronic copies of patient EOB or ERA statements detailing the treatment received and the payments made by the payer, such as insurance companies. The practice must then manually reassociate the EOB/ERA with the payment received from the payer to ensure they correspond. In many instances, large payers may remit batch payments that include multiple payments for multiple patients, which can make the reassociation process time-consuming and prone to errors. After a payment and EOB/ERA has been reassociated, the practice must then manually post the insurance payment in the patient ledger of the practice management system. This process can take several days to a week to complete manually. This process may further be prone to errors and allow for fraud if medical practice employees do not enter the reassociated payment information correctly into the patient ledger of the practice management system.

Solutions are therefore needed to provide an automated payer remittance, EOB/ERA reassociation, and payment posting process. Providing an automated payer remittance, EOB/ERA reassociation, and payment posting process may save time, reduce errors, and prevent fraud throughout this process. Dental and physician practices have highly fragmented practice management systems with dozens of providers. This creates a “last mile” problem in which dental and physician practices are not able to automate the payment posting process because of the number of integrations with practice management systems that are needed. To avoid the “last mile” problem of automatic payment posting resulting from the large number of integrations that could be needed with the variety of practice management systems, such solutions should be agnostic to any specific practice management system. Such solutions may allow the payment posting technology to be used with any variety of practice management systems without requiring direct integration with specific practice management systems. Such solutions should provide a more efficient, accurate, and secure process for payment remittance, EOB/ERA reassociation, and payment posting.

### SUMMARY

Methods for providing automated payment processing and posting are provided. The methods may include receiving a payment from a third party, receiving a payment statement from the third party, automatically reassociating the payment and the payment statement based on patient data in the payment and the payment statement, and automatically posting, by a robotic process automation platform, the reassociated payment and payment statement to a patient ledger of a practice management software.

In some embodiments, reassociating the payment and the payment statement may further include matching the payment with the payment statement.

In some embodiments, receiving the payment may further include receiving an electronic funds transfer.

In some embodiments, the robotic process automation platform may include a platform configured to execute repeatable data gathering actions.

In some embodiments, the robotic process automation platform may include a platform configured to execute repeatable input actions.

In some embodiments, the payment may include a batch payment corresponding to a plurality of claims.

In some embodiments, the payment may include a single payment corresponding to a single claim.

In some embodiments, the methods may further comprise automatically capturing the payment from the third party.

In some embodiments, the practice management software may include a computerized records management platform.

In some embodiments, the patient ledger may include a record of financial transactions associated with a patient.

In some embodiments, a system may be provided for automated payment processing and posting. The system may include at least one processor and at least one memory storing instructions that, when executed by the at least one processor, cause the at least one processor to receive a payment from a third party, receive a payment statement from the third party, automatically reassociate the payment and the payment statement based on patient data in the payment and the payment statement, and automatically post, by a robotic process automation platform, the reassociated payment and payment statement to a patient ledger of a practice management software.

In some embodiments, reassociating the payment and the payment statement may include matching the payment with the payment statement.

In some embodiments, receiving the payment may include receiving an electronic funds transfer.

In some embodiments, the robotic process automation platform may include a platform configured to execute repeatable data gathering actions.

In some embodiments, the robotic process automation platform may include a platform configured to execute repeatable input actions.

In some embodiments, the payment may include a batch payment corresponding to a plurality of claims.

In some embodiments, the payment may include a single payment corresponding to a single claim.

In some embodiments, the instructions may further include automatically capturing the payment from the third party.

In some embodiments, the practice management software may include a computerized records management platform.

In some embodiments, the patient ledger may include a record of financial transactions associated with a patient.

The above and other aspects and their implementations are described in greater detail in the drawings, the descriptions, and the claims.

### BRIEF DESCRIPTION OF THE DRAWINGS

The accompanying drawings, which are incorporated in and constitute a part of this specification, illustrate disclosed embodiments and, together with the description, serve to explain the disclosed embodiments.

FIG. 1 depicts an exemplary conventional payment reassociation and posting process, in accordance with disclosed embodiments.

FIG. 2 depicts an exemplary automated payment reassociation and posting process, in accordance with disclosed embodiments.

FIG. 3 depicts an example of a computing device, in accordance with disclosed embodiments.

FIG. 4 depicts a revenue cycle management process, in accordance with disclosed embodiments.

FIG. 5 depicts an automated portion of the revenue cycle management process of FIG. 4, in accordance with disclosed embodiments.

FIG. 6 depicts a flowchart of a process for providing automated payment processing and posting, in accordance with disclosed embodiments.

### DETAILED DESCRIPTION

Reference will now be made in detail to exemplary embodiments, discussed with regard to the accompanying drawings. In some instances, the same reference numbers will be used throughout the drawings and the following description to refer to the same or like parts. Unless otherwise stated, technical and/or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. For example, unless otherwise indicated, method steps disclosed in the figures may be rearranged, combined, or divided without departing from the envisioned embodiments. Similarly, additional steps may be added or steps may be removed without departing from the envisioned embodiments. Thus, the materials, methods, and examples are illustrative only and are not intended to be necessarily limited.

The disclosed embodiments address drawbacks in conventional techniques through the use of an automated payment reassociation and posting system for dental and physician practices. Conventional processes for payment reassociation and posting are time consuming, error prone, and may allow for fraud throughout the process. An automated payment reassociation and posting system may improve existing techniques by enabling automatic payment remittance and EOB/ERA to payment reassociation. Such solutions may also enable automatic payment posting to a patient ledger within the practice's practice management software. Such solutions may reduce clerical errors at the practice level and mitigate fraud for the dental or physician practice. Accordingly, such solutions may be more efficient, more accurate, and more secure than conventional processes.

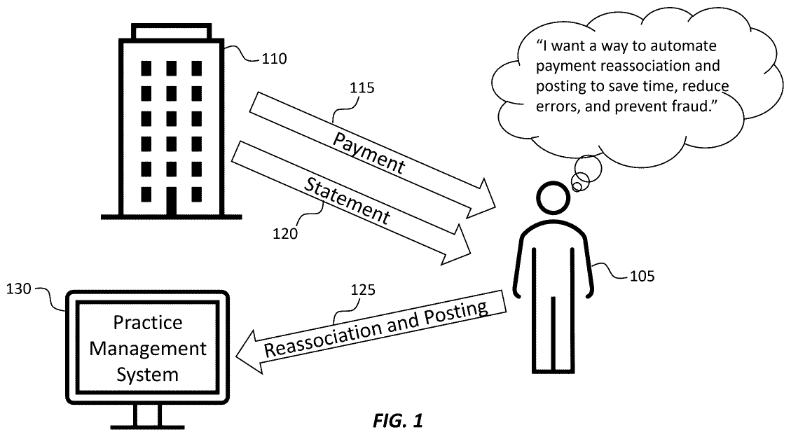

FIG. 1 is an illustration of a situation involving healthcare worker 105 expressing a desire for a way to automate the payment reassociation and posting process to save time, reduce errors, and prevent fraud. Healthcare worker 105 may be a person associated with a dental or physician practice. For example, healthcare worker 105 may be a dentist or physician, a technician, an office manager, an accountant, or any other person associated with a dentist or physician practice. As illustrated in FIG. 1, healthcare worker 105 may receive payment 115 from payer 110 and may separately receive statement 120 from payer 110. Payer 110 may include insurance organizations, health plan providers, or other organizations that may pay claims for medical services provided to a patient. Payment 115 may include a reimbursement for a claim submitted by healthcare worker 105 for medical services provided to a patient. Statement 120 may include an explanation of benefits (“EOB”) statement and/or an electronic remittance advice (“ERA”) statement. Statement 120 may explain how insurance benefits were applied to a medical claim along with patient and service information. In the current situation, healthcare worker 105 may have to manually reassociate, as shown at reassociation and posting 125, payment 115 with a corresponding statement 120 received from payer 110 to confirm that payer 110 provided the proper reimbursement amount for medical services provided to a patient. Healthcare worker 105 may then manually post the payment amounts and supporting documentation to practice management system 130 to record and document proper payment from payer 110. Practice management system 130 may be a computerized healthcare software to manage patient records, billing, and claims processing.

Although FIG. 1 depicts one payment 115, one statement 120, and one payer 110, these are merely exemplary, and healthcare worker 105 may receive many payments and statements from many payers each day. Further, payment 115 may include batch payments that may include payments for many different patients in a single transaction. Payment 115 and statement 120 also may not be received at the same time or in the same format (i.e., payment 115 may be received electronically while statement 120 may be received through paper copies, or vice versa). It may take several days to a week for healthcare worker 105 to manually reassociate and post payment 115 with the correct corresponding statement 120 to confirm the correct reimbursement amounts are received for each patient and then post the proper payment amounts to practice management system 130. Manually reassociating payment 115 with statement 120 and posting the processed payments to practice management system 130 may be time-consuming, error prone, and may allow for fraud.

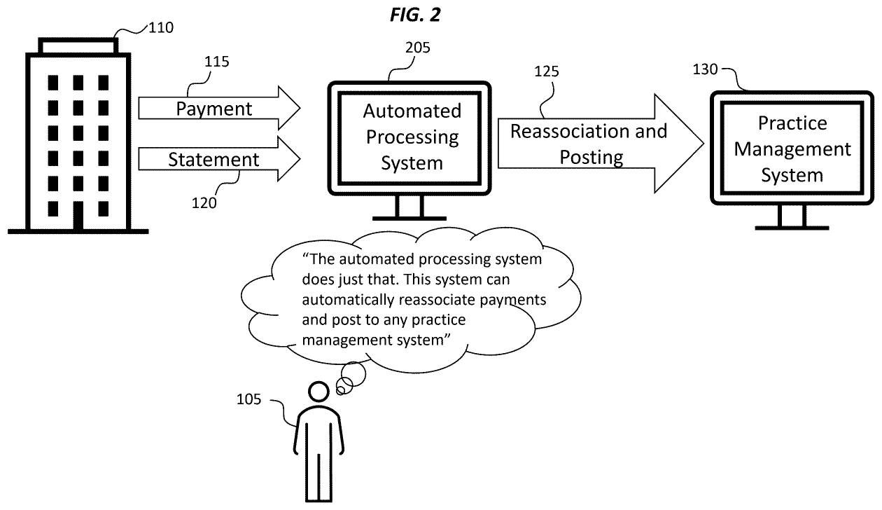

FIG. 2 is an illustration of a situation involving healthcare worker 105 expressing a satisfied experience with automated processing system 205. FIG. 2 illustrates components of FIG. 1, the descriptions of which are incorporated herein by reference. Automated processing system 205 may receive payment 115 and statement 120 from payer 110. Automated processing system 205 may automatically reassociate and post 125 each payment 115 with the corresponding statement 120. Automated processing system 205 may then automatically post the reimbursement amounts to practice management system 130. Automated processing system 205 may be agnostic to practice management systems and may therefore automatically post reimbursement amounts to any practice management system 130. By using automated processing system 205, healthcare worker 105 may not need to manually reassociate and post payment amounts to practice management system 130. This may save time, reduce errors, and prevent fraud during the reassociation and posting of payment 115.

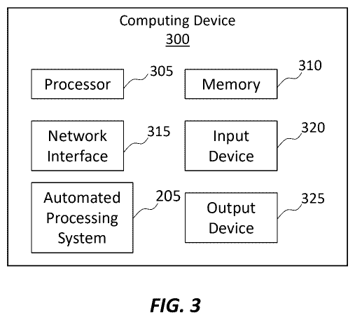

FIG. 3 depicts a computing device 300 including automated processing system 205, according to embodiments of the present disclosure. Computing device 300 may be a variety of different types of computing devices capable of developing, storing, analyzing, and/or executing software code. For example, computing device 300 may be a personal computer (e.g., a desktop or laptop), an IoT device (e.g., sensor, smart home appliance, connected vehicle, etc.), a server, a mainframe, a vehicle-based or aircraft-based computer, a virtual machine (e.g., virtualized computer, container instance, etc.), or the like. Computing device 300 may be a handheld device (e.g., a mobile phone, a tablet, or a notebook), a wearable device (e.g., a smart watch, smart jewelry, an implantable device, a fitness tracker, smart clothing, a head-mounted display, etc.), an IoT device (e.g., smart home devices, industrial devices, etc.), or various other devices capable of processing and/or receiving data. Computing device 300 may operate using a Windows™ operating system, a terminal-based (e.g., Unix or Linux) operating system, a cloud-based operating system (e.g., through AWS™, Azure™, IBM Cloud™, etc.), or other types of non-terminal operating systems.

Computing device 300 may include at least one processor 305 and at least one memory 310. Processor 305 may include, for example, central processing units (CPUs), graphics processing units (GPUs), digital signal processors (DSPs), field programmable gate arrays (FPGAs), integrated circuits, microcontrollers, microchips, microprocessors, or other units suitable for executing instructions or performing logic operations. Processor 305 may include a single-core or multiple-core processor (e.g., dual-core, quad-core, or with any desired number of cores). Processor 305 may provide the ability to execute, run, control, manage, or store multiple processes, applications, or programs. Furthermore, according to some embodiments, processor 305 may be from the family of processors manufactured by Intel®, AMD®, Qualcomm®, Apple®, NVIDIA®, or the like. Processor 305 may also be based on the ARM architecture, a mobile processor, or a graphics processing unit, etc. The disclosed embodiments are not limited to any type of processor configured in the computing device 300.

Memory 310 may include a non-transitory computer-readable medium that may store instructions. Memory 310 may include, for example, volatile memory, non-volatile memory, flash drives, caches, registers, hard drives, disks, an optical data storage medium, a physical medium with patterns, random access memory (RAM), read-only memory (ROM), programmable read-only memory (PROM), erasable programmable read-only memory (EPROM), compact disc read-only memory (CD-ROM), digital versatile discs (DVDs), non-volatile random-access memory (NVRAM), or networked versions thereof. The disclosed embodiments are not limited to software programs or devices configured to perform dedicated tasks. For example, memory 310 may store a single program, such as a user-level application, that performs the functions of the disclosed embodiments, or may include multiple software programs. Additionally, processor 305 may in some embodiments execute one or more programs (or portions thereof) remotely located from the computing device 300. Furthermore, memory 310 may include one or more storage devices configured to store data (e.g., machine learning data, training data, algorithms, etc.) for use by the programs, as discussed further below.

Computing device 300 may further include at least one network interface 315 (e.g., a network card, a modem, and/or any other device that may be configured to provide data communication via a network), one or more input devices 320 (e.g., a keyboard, a mouse, a touch screen, a joystick, a touch pad, one or more buttons, a microphone, a sensor, and/or any other device configured to detect and/or receive input), and/or one or more output devices 325 (e.g., a display (e.g., a light-emitting diode (LED) display, a liquid-crystal display (LCD), an organic light-emitting diode (OLED) display, or a dot-matrix display), a screen, a touch screen, a headphone, a speaker, a light indicator, a light source, a device configured to provide tactile cues, a vibrator, and/or any other device configured to provide output).

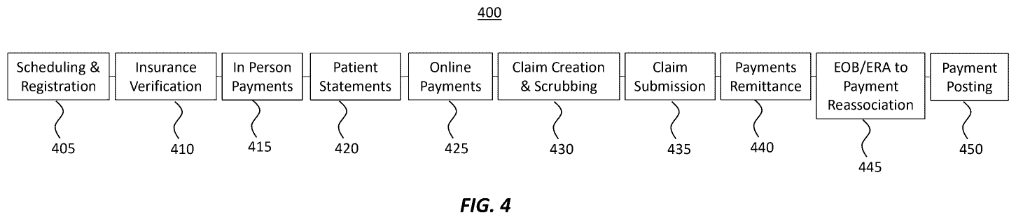

FIG. 4 depicts a Revenue Cycle Management (“RCM”) process 400, in accordance with disclosed embodiments. RCM process 400 may include a process to enable healthcare practices, such as dental and physician practices, to track and manage payments for providing medical services. RCM process 400 may enable accurate management of patient interactions from scheduling an appointment through final payment. RCM process 400 may also ensure that appropriate patient information is collected and managed, patients are properly billed for services provided, and third-party payers, such as insurance companies, are alerted in a timely manner so that payments can be collected efficiently. Reference will now be made to steps 405 through 435 of RCM process 400, which provide background of the general revenue cycle management process.

As depicted in FIG. 4, step 405 of RCM process 400 may include scheduling and registration. Step 405 may include collecting patient data, such as contact and demographic information, insurance coverage, medical history information, consent forms, and appointment scheduling. Scheduling may be completed manually or automated through a patient intake system. Step 410 of RCM process 400 may include insurance verification. Step 410 may include confirming a patient's insurance coverage and benefits to enable accurate billing and reimbursement for patient services. Step 415 of RCM process 400 may include in-person payments. Step 415 may include collecting payments from patients in person as necessary based on the patient's identified insurance coverage and the medical services provided to the patient. Step 420 of RCM process 400 may include patient statements. Step 420 may include generating and delivering financial statements to patients that may provide a breakdown of medical services rendered, associated costs, and the patient's financial responsibility for such services after insurance adjustments. Step 425 of RCM process 400 may include online payments. Step 425 may include collecting online payment from a payer, for example through an online portal associated with the dental or physician practice. Step 430 of RCM process 400 may include claim creation and scrubbing. Step 430 may include compiling claims to be submitted to an insurance company, ensuring each claim accurately reflects the patient information and medical services provided, and confirming the claims meet the requirements of the relevant insurance company. Step 435 of RCM process 400 may include claim submission. Step 435 may include submitting the compiled claims to an insurance company for reimbursement.

Reference will now be made to steps 440 through 450 of RCM process 400. Steps 440 through 450 of current revenue cycle management techniques may not be fully automated for dental and physician practices, as disclosed herein with respect to FIG. 4. Step 440 of RCM process 400 may include payment remittance. Step 440 may include receiving payment from an insurance company, or other payer, to cover a claim submitted in step 435 of RCM process 400. Payer payments may be received in different formats, such as through paper checks, EFT transfers to bank accounts, by credit card, or by any other means of sending and receiving payments. In some embodiments, payer payments may be made as a single payment in which the payment is associated with the cost of medical services provided to one patient. In other embodiments, payer payments may be made as a batch payment in which one payment is associated with the cost of medical services provided to many patients. Step 445 of RCM process 400 may include EOB/ERA to payment reassociation. Step 445 may include receiving paper or electronic copies of patient EOB and/or ERA statements. The EOB and ERA statements may detail the treatment received by the patient and the payments received by the payer, such as an insurance company. Step 445 may include reassociating the EOB and/or ERA with the payment that was received from the payer at step 440 of RCM process 400. Reassociating the EOB and/or ERA with the payment may include matching patient identifying information (such as patient name, claim number, etc.) from the payment information and the EOB statement and/or ERA statement. Step 445 may confirm that the payment received from the payer at step 440 of RCM process 400 matches the information from the EOB and/or ERA statements. Step 450 of RCM process 400 may include payment posting. Step 450 may include posting the insurance payments in the patient ledger of the practice management system to document and record the accurate payment of the services rendered for the patient. The patient ledger of the practice management system may record financial transactions associated with a particular patient of the medical practice to provide financial accounting for medical services provided to the patient. Current revenue cycle management techniques may require a medical practice to manually input reassociated payment information into the patient ledger of the practice management system. Such techniques may be time inefficient and error prone, and they may also allow for fraud.

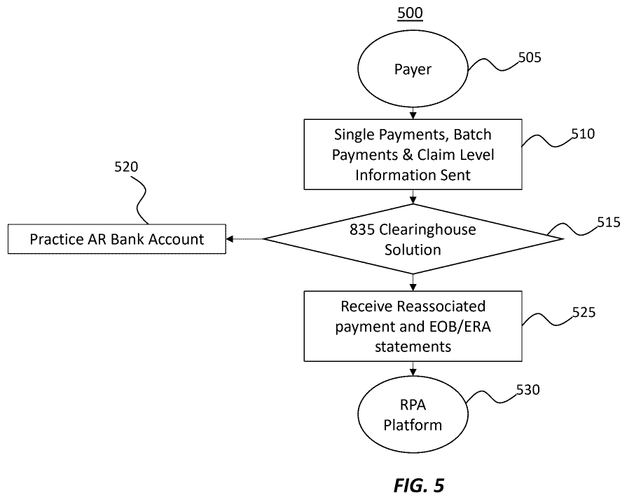

FIG. 5 depicts a process 500 for automating steps 440, 445, and 450 of RCM process 400. Automating steps 440, 445, and 450 of RCM process 400 may lead to more accurate record keeping for dental and physician practices and may reduce errors and prevent fraud that may occur, e.g., when steps 440, 445, and 450 are not completed in accordance with disclosed embodiments. Accordingly, automating steps 440, 445, and 450 may provide a more accurate, efficient, and secure payment processing and posting system.

At step 505 of process 500, payers may process provider claims. A payer may include an organization, such as a health plan provider or insurance organization, that may set rates, collect payments, process claims, and pay provider claims. A claim may include a request for payment from a provider to a health insurer for medical services provided to a patient.

Step 510 of process 500 may include receiving batch payments or single payments and claim level information (such as a bill classification code, patient control number, statement date, claim control number, or other claim-related information) for provider claims. A single payment may include a payment that corresponds to a single claim for medical or dental services provided to a single patient. A batch payment may include a single cumulative deposit from a payer to a provider which may include multiple, separate claim amounts for medical or dental services provided to many patients. In some embodiments, single and/or batch payments may be received in different formats, such as through paper checks, EFT transfers to a bank account, through credit card payments, or through any other form of sending and receiving payments. Step 510 of process 500 may further include receiving EOB and/or ERA statements with the batch or single payments. An EOB statement may include an explanation of how insurance benefits were applied to a claim along with patient and service information. An ERA statement may include an electronic version of the EOB statement. In some embodiments, the payments and EOB and/or ERA statements may be received in a paper format. In other embodiments, the payments and EOB and/or ERA statements may be received in electronic format. The payments and EOB and/or ERA statements may be received through an 835 clearinghouse. An 835 clearinghouse may refer to one or more entities that facilitate the electronic exchange of healthcare information between providers, payers, and other stakeholders. An 835 clearinghouse may also act as an intermediary between a provider and an insurance company. For example, in some embodiments, an 835 clearinghouse may ensure that claims are accurately submitted to an insurance company from a healthcare provider and may also enable accurate and efficient payment of claims from the insurance company to the healthcare provider.

Step 515 of process 500 may include an automated capture of electronic payer remittance and an automated reassociation of the remitted payment(s) with the EOB and/or ERA statements. To automate the capture the electronic payer remittance, step 515 may include receiving payer remittance in electronic format through a secure channel setup for electronic data interchange (EDI). EDI setup may include, e.g., using an EDI software that supports the ANSI X12 835 transaction set, using custom EDI parsers operating based on programming code (e.g., PYTHON, JAVA, etc.), and/or utilizing a cloud-based EDI service (e.g., AWS EDI, AZURE LOGIC APPS, etc.) for EDI integration. Securing the channel may include, e.g., implementing Secure FTP (SFTP) or Secure HTTP (HTTPS) for data transmission to/from the channel, using a Virtual Private Network (VPN) for data exchange, or using encryption (e.g., TLS/SSL) for data exchange. Step 515 may, e.g., be conducted by an 835 clearinghouse that may ensure that claims are accurately paid by the payer to the healthcare provider. For example, the 835 clearinghouse may act as an intermediary between the payer (e.g., an insurance provider) and the medical services provider (e.g., a dentist's office, a doctor's office, or another medical office). The payer may transmit EOB and/or ERA statements directly to the 835 clearinghouse or to another secure channel setup for EDI.

Step 515 may further include automatically reassociating the payment with the EOB and/or ERA statements. For example, step 515 may include matching the single or batch payments with the EOB and/or ERA statements to ensure that the proper payments were received from the payer for the services rendered by the healthcare provider. For example, the EOB and/or ERA statements may include patient and policy information, provider information, service details, billing codes, dates of services, amounts charged by the provider, amounts covered by the payer, deductible details, and any other information related to the medical or dental service provided to a patient. The single or batch payments may also include patient and policy information, provider information, billing codes, dates of services, and other identifying information. Matching the single or batch payments with the EOB and/or ERA statement may include matching the identifying information included in the EOB and/or ERA statement with the identifying information included in the single or batch payment. Step 515 may provide an automated process for matching and confirming the accuracy of the payments and the EOB and/or ERA statements. For example, if the EOB and/or ERA statement includes payment information that equals the received payment from the payer, then it may be determined that the payer made an accurate payment for the medical services provided to a patient. If the EOB and/or ERA statement identifies a payment amount that differs from the payment received from the payer, then it may be determined that the payer did not make an accurate payment for the medical services provided to a patient. As another example, the patient and policy information may include a reassociation trace number (RTN), which may refer to a unique identifier included in the payer remittance, EOB statement, and ERA statement, and matching may include comparing the RTN across the payer remittance data received and the EOB and/or ERA statements. Further, matching may also include parsing the payer remittance data received and the EOB and/or ERA statements to locate and determine identifiers provided therein, such as, e.g., the RTN or other patient or policy information which may be unique to a particular patient. Parsing the payer remittance data received and the EOB and/or ERA statements (or other 835 files) may include, e.g., using libraries or tools such as EDI NOTEPAD or an EDI translator, developing custom parsers using libraries such as, e.g., PYX12 (for PYTHON) or SMOOKS (for JAVA), utilizing a cloud-based services (e.g., AWS LAMBDA) and custom parsing scripts, or otherwise configuring a parser (e.g., parsing technology or parsing service) based on known or expected locations of particular data (e.g., the RTN or another unique identifier) within the EOB/ERA statements, and/or based on triangulation techniques using pixel data based on the known or expected locations of particular data in the EOB/ERA statements (or other 835 files). As a non-limiting example, automatically reassociating based on the RTN may include any or all of implementing a database lookup system to match the RTN from the payer remittance data and the EOB/ERA statements, using machine learning models to predict and match payment remittance data (e.g., RTN associated with payment remittance data) and the EOB/ERA statement data (e.g., RTN associated with the EOB/ERA statements), or otherwise providing rule-based programming for automating the reassociation process.

It will be understood that a robust database system may also be utilized during the reassociation process, such that the captured data may be stored and managed efficiently and effectively (e.g., to support efficient querying of the captured data and/or reporting based on the querying). Such a database system may include, e.g., relational databases (e.g., MYSQL, PRESGRESQL, or SQL SERVER), non-relational databases (e.g., NOSQL, MONGODB, CASSANDRA), and/or cloud-based database services (e.g., AMAZON RDS, AZURE SQL DATABASE, GOOGLE CLOUD FIRESTONE). It will further be understood that data management techniques and solutions (e.g., based on one or more of data warehousing for large-scale data storage and analysis, extract-transform-load (ETL) tools, or custom data management scripts) may also be included during the reassociation process.

At step 520 of process 500, funds may be transferred (e.g., via the 835 clearinghouse, directly, or via another third-party) to the provider bank account (e.g., after the reassociation process and based on an accurate reassociation). Funds may be transferred through an electronic funds transfer (“EFT”). An EFT may include a transfer of funds from one account to another across a computerized platform without the direct intervention of bank staff.

Step 525 of process 500 may include electronically receiving the reassociated payment information by a robotic process automation (“RPA”) system. The RPA system may include software designed to execute repeatable data gathering or input actions within graphic user interfaces or software platforms. For example, the RPA system may use intelligent automation techniques to perform repetitive data gathering and input actions. The RPA system may use virtual robots (bots) to automate repetitive tasks. For example, RPA software robots may perform a variety of tasks, such as data entry, transaction processing, report generation, and other repetitive tasks. The RPA software robots may interact with an interface in the same way that a human would, to record and mimic human actions. For example, the RPA software robots may interact with a healthcare provider's patient management software. The RPA software robots may complete data entry tasks, such as inputting the reassociated payment and payment information into the healthcare provider's patient management software. In some embodiments, the practice management software may include a computerized records management platform utilized by a healthcare provider to manage administrative and operational tasks. The patient ledger of the practice management software may include a record of financial transactions associated with a patient. For example, the patient ledger may include records of payments received from the patient, payments received from third parties, such as insurance providers, and outstanding payments. The RPA system may be agnostic to specific practice management software which may allow the RPA system to automate payment posting to a patient ledger. For example, because dental and physician practices may use a variety of types of patient management software, the RPA system may be configured to post payments to the patient ledger of any patient management software used by the dental or physician practice, as disclosed herein.

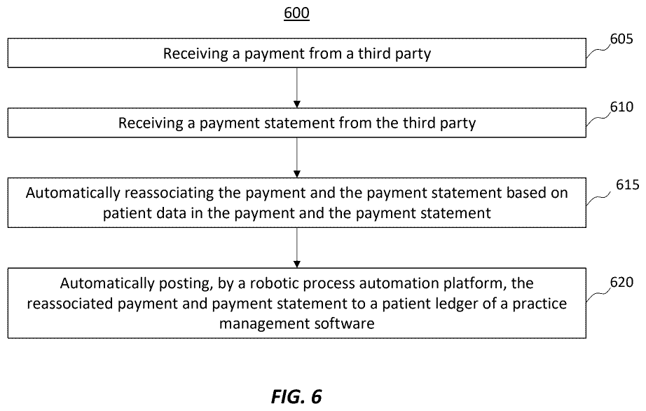

Step 530 of process 500 may include posting payment information by the RPA system to a practice management software patient ledger. A practice management software may include a computerized healthcare platform for managing patient records, communications, scheduling, billing, and/or claims processing. A patient ledger may include a section of a patient file that may contain all financial transactions related to the patient account as well as a record of the services provided to the patient. The RPA system may automatically post the payment information that was reassociated in step 515 of process 500 to the patient ledger of the practice management system, as disclosed herein with respect to FIG. 6. The RPA system may be agnostic to the type of practice management system used by a healthcare provider, which may allow the RPA system to post payment information to any practice management system used by a healthcare provider. For example, the RPA system may receive the reassociated payment information and be configured to post the payment information to any practice management system used by a healthcare provider.

The RPA system may be enabled to automate payment posting to a patient ledger by, e.g., configuring the system to integrate with various practice management software (e.g., by using an application programming interface (API) or via other integration methods). API integration may include, e.g., using restful APIs, Simple Object Access Protocol (SOAP)-based web services, or middleware solutions (e.g., MULESOFT, APACHE CAMEL). Automated payment posting may include, e.g., using custom scripts to post data directly into a practice management software, using RPA tools (e.g., UIPATH, BLUE PRISM, AUTOMATION EVERYWHERE), implementing batch processing systems to update the practice management software, e.g., after the reassociation process, and/or using cloud-based RPA services (e.g., AWS ROBOMAKER, AZURE AUTOMATION). Further, the RPA system may also be trained and improved over time. For example, the RPA system may implement machine learning models to continuously improve RPA performance. As another example, the RPA system may use feedback loops to adjust and optimize RPA workflows. As yet another example, the RPA system may integrate with artificial intelligence services (e.g., IBM WATSON, GOOGLE AI) to further improve its learning capabilities and performance.

FIG. 6 depicts a process 600 for providing automated payment processing and posting. Although FIG. 6 shows example blocks of process 600, in some implementations, process 600 may include additional blocks, fewer blocks, different blocks, or differently arranged blocks than those depicted in FIG. 6. Additionally, or alternatively, two or more of the blocks of process 600 may be performed in parallel.

Step 605 of process 600 may include receiving a payment from a third party. The third party may include a payer, such as an insurance provider or health plan provider responsible for providing payment for medical services provided to a patient. In some embodiments, the payment may include a single payment that may correspond to a single claim for medical treatment provided to a single patient. In other embodiments, the payment may include a batch payment. A batch payment may include a single payment from the third party that may correspond to a plurality of claims for medical treatments provided to a plurality of patients. For example, the third party (such as the insurance provider or health plan provider) may transmit a single payment that may reimburse a health care provider for medical services provided to a plurality of patients. In some embodiments, the batch payment may include a single payment for multiple appointments or medical services provided to a single patient over a period of time. In other embodiments, the batch payment may include a single payment for appointments and medical services provided to multiple patients over a period of time. In some embodiments, the payment may be received through an electronic transfer of funds. In other embodiments, the payment may be received through paper checks, credit card payments, or any other payment method.

Step 610 of process 600 may include receiving a payment statement from the third party. The payment statement may include an EOB statement and/or an ERA statement. An EOB statement may include an explanation of how insurance benefits were applied to a claim along with patient and service information. An ERA statement may include an electronic version of the EOB. In some embodiments, the EOB statement and/or ERA statement may be received in a paper format. In other embodiments, the EOB statement and/or the ERA statement may be received in electronic format. In some embodiments, the EOB statement and/or the ERA statement may be received at the same time as the payment. In other embodiments, the EOB statement and/or the ERA statement may be received at a different time as the payment.

Step 615 of process 600 may include automatically reassociating the payment and the payment statement. Reassociating the payment and the payment statement may include matching payment information from the EOB statement and/or ERA statement with the payments received from the third party (as described in more detail with reference to FIG. 5 herein). For example, the single payment or batch payment received from the third party may include patient information, such as a patient name, claim number, or other patient identifying information. The EOB statement and/or ERA statement may also include the patient name, claim number, or other patient identifying information. Reassociating the payment and payment statement may include automatically matching the patient identifying information from the payment information and the EOB statement and/or ERA statement. Reassociating the payment and payment statement may identify any errors in the payment from the third party. For example, reassociating the payment and payment statement may identify whether the third party over paid or underpaid the amount provided in the payment statement. Reassociating the payment statement and the payment may be done automatically, without human intervention.

Step 620 of process 600 may include automatically posting, by a robotic process automation platform, the reassociated payment and payment statement to a patient ledger of a practice management software (as described in more detail with reference to FIG. 5 herein). A robotic process automation platform may include a platform configured to execute repeatable data gathering actions, such as extracting data, filling in forms, moving files, generating reports, processing invoices, and other repetitive, rule-based tasks. For example, the robotic process automation platform may include a platform that may be configured to execute repeatable input actions. The robotic process automation system may use virtual robots, or “bots,” to automate repetitive tasks. For example, robotic process automation software robots may perform a variety of tasks, such as data entry, transaction processing, report generation, and other repetitive tasks. The robotic process automatic software robots may interact with an interface in the same way that a human would, to record and mimic human actions. For example, the robotic process automation software robots may interact with a healthcare provider's patient management software. The robotic process automation software robots may complete data entry tasks, such as inputting the reassociated payment and payment information into the healthcare provider's patient management software. In some embodiments, the practice management software may include a computerized records management platform utilized by a healthcare provider to manage administrative and operational tasks. The patient ledger of the practice management software may include a record of financial transactions associated with a patient. For example, the patient ledger may include records of payments received from the patient, payments received from third parties, such as insurance providers, and outstanding payments.

Robotic process automation (RPA) software robots can be configured to interact with a variety of interfaces, therefore the robotic process automation software may be deployed across a wide variety of patient management software. Healthcare providers may use one or more of dozens of patient management software platforms, but the robotic process automation software robots may be configured to interact with any of these patient management platforms to complete the repetitive task of inputting reassociated payments and payment information into the patient management software. Configuring an RPA software robot to interact with various patient management software and a different interfaces thereof may include various techniques, such as, e.g., rule-based programming, pixel triangulation techniques, integrating artificial intelligence into the RPA platform, integrating APIs using restful APIs or SOAP-based web services, screen scraping techniques (e.g., optical character recognition (OCR) and image recognition, which may be particularly useful for legacy systems), using direct database access and/or middleware solutions (e.g., MULESOFT, APACHE CAMEL), and/or using RPA platforms (e.g., UIPATH, BLUE PRISM, or AUTOMATION ANYWHERE). By combining these methods, an RPA bot can effectively automate tasks across different patient management systems, ensuring efficiency and accuracy.

For example, the robotic process automation software robots may be configured to follow rule-based programming that direct the software robots to input data into the patient management software. Such rule-based programming may include programming that directs the robotic process automation software robots to log into specific applications, such as the patient management software, navigate interfaces within the applications, input data (such as reassociated data between payments and payment statements), and other tasks related to posting payment information to the patient management software. The robotic process automation software robots may be configured to interact and integrate with various systems, such as the patient management system and systems that receive the reassociated payment and payment statement information. This may allow the robotic process automation software robots to input information into a variety of patient management systems.

It is to be understood that the disclosed embodiments are not necessarily limited in their application to the details of construction and the arrangement of the components and/or methods set forth in the description and/or illustrated in the drawings and/or the examples. The disclosed embodiments are capable of variations, or of being practiced or carried out in various ways.

The disclosed embodiments may be implemented in a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may include copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, to perform aspects of the present disclosure.

Aspects of the present disclosure are described herein with reference to flowchart illustrations and/or block diagrams of methods, apparatus (systems), and computer program products according to embodiments of the present disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus, or other device to produce a computer implemented process, such that the instructions which execute on the computer, other programmable apparatus, or other device implement the functions/acts specified in the flowchart and/or block diagram block or blocks.

The flowcharts and block diagrams in the Figures illustrate the architecture, functionality, and operation of possible implementations of systems, methods, and computer program products according to various embodiments of the present disclosure. In this regard, each block in the flowcharts or block diagrams may represent a software program, segment, or portion of code, which includes one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant virtualization platforms, virtualization platform environments, trusted cloud platform resources, cloud-based assets, protocols, communication networks, security tokens and authentication credentials, and code types will be developed, and the scope of these terms is intended to include all such new technologies a priori.

It is appreciated that certain features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable sub combination or as suitable in any other described embodiment of the present disclosure. Certain features described in the context of various embodiments are not to be considered essential features of those embodiments unless the embodiment is inoperative without those elements.

Although the present disclosure has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

What is claimed is:

1. A computer implemented method for providing automated payment processing and posting, the method comprising:
receiving a payment from a third party;
receiving a payment statement from the third party;
automatically reassociating the payment and the payment statement based on patient data in the payment and the payment statement; and
automatically posting, by a robotic process automation platform, the reassociated payment and payment statement to a patient ledger of a practice management software.

2. The computer implemented method of claim 1, wherein reassociating the payment and the payment statement further includes matching the payment with the payment statement.

3. The computer implemented method of claim 1, wherein receiving the payment further includes receiving an electronic funds transfer.

4. The computer implemented method of claim 1, wherein the robotic process automation platform includes a platform configured to execute repeatable data gathering actions.

5. The computer implemented method of claim 4, wherein the robotic process automation platform includes a platform configured to execute repeatable input actions.

6. The computer implemented method of claim 1, wherein the payment includes a batch payment corresponding to a plurality of claims.

7. The computer implemented method of claim 1, wherein the payment includes a single payment corresponding to a single claim.

8. The computer implemented method of claim 1, wherein the method further comprises automatically capturing the payment from the third party.

9. The computer implemented method of claim 1, wherein the practice management software includes a computerized records management platform.

10. The computer implemented method of claim 1, wherein the patient ledger includes a record of financial transactions associated with a patient.

11. A system for providing automated payment processing and posting, the system comprising:
a memory storing instructions; and
at least one processor configured to execute the instructions to:
receive a payment from a third party;
receive a payment statement from the third party;
automatically reassociate the payment and the payment statement based on patient data in the payment and the payment statement; and
automatically post, by a robotic process automation platform, the reassociated payment and payment statement to a patient ledger of a practice management software.

12. The system of claim 11, wherein reassociating the payment and the payment statement further includes matching the payment with the payment statement.

13. The system of claim 11, wherein receiving the payment further includes receiving an electronic funds transfer.

14. The system of claim 11, wherein the robotic process automation platform includes a platform configured to execute repeatable data gathering actions.

15. The system of claim 14, wherein the robotic process automation platform includes a platform configured to execute repeatable input actions.

16. The system of claim 11, wherein the payment includes a batch payment corresponding to a plurality of claims.

17. The system of claim 11, wherein the payment includes a single payment corresponding to a single claim.

18. The system of claim 11, wherein the instructions further include automatically capturing the payment from the third party.

19. The system of claim 11, wherein the practice management software includes a computerized records management platform.

20. The system of claim 11, wherein the patient ledger includes a record of financial transactions associated with a patient.
